(19) United States
(12) Patent Application Publication
(10) Pub. No.: US 2008/0050333 A1
(43) Pub. Date: Feb. 28, 2008

(54) OIL-IN-WATER DISPERSION

(75) Inventors: Cyril Lemoine, Saint Cyr I'Ecole (FR); Aldo Monello, Savigny Sur Orge (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/845,169

(22) Filed: Aug. 27, 2007

Related U.S. Application Data
(60) Provisional application No. 60/841,206, filed on Aug. 31, 2006.

(30) Foreign Application Priority Data
Aug. 25, 2006 (FR) 06 53472

(51) Int. Cl. A61K 8/81; A61K 8/04; A61Q 5/00

(52) U.S. Cl. 424/70.16

(57) ABSTRACT

A composition in the form of an oil-in-water dispersion containing:
at least 60% by weight of water, relative to the total weight of the composition;
at least one glyceryl acrylate polymer;
at least one homopolymer of a monomer having a sulphonic group;
at least one volatile oil in an amount of less than or equal to 20% by weight, relative to the total weight of the composition; and
0 to 0.5% of at least one non-volatile oil.

### REFERENCE TO PRIOR APPLICATIONS

This application claims priority to U.S. provisional application 60/841,206 filed Aug. 31, 2006, and to French patent application 0653472 filed Aug. 25, 2006, both incorporated herein by reference.

### FIELD OF THE INVENTION

The present invention relates to a composition in the form of an oil-in-water dispersion comprising a blend of polymers and to a treatment method for keratin materials.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cosmetic skincare compositions are often in the form of water-in-oil or oil-in-water emulsions or multiple water-in-oil-in-water emulsions in order to obtain a pleasant sensory effect on the skin. This is because the application of an aqueous solution on the skin is not pleasant for the user and has a tendency to dry the skin as opposed to oils such as apricot kernel oil that nourish the skin and avoid drying it. In addition, an aqueous solution applied to the skin does not spread correctly if it is too fluid and therefore makes it impossible to obtain a homogeneous deposition on the skin.

Moreover, emulsions have drawbacks. Certain surfactants required for the stability of the emulsion, such as fatty acid soaps, for example stearic acid soaps, are uncomfortable for the skin because they cause irritation and are also drying.

In addition, when the emulsions contain hydrophilic active agents, the presence of non-volatile fatty substances such as non-volatile oils (vegetable oils, oils of mineral origin, non-volatile silicone oils) or waxes form a residual film on the skin which prevents good penetration of the active agent which is retained on the surface of the skin. Furthermore, the presence of emulsifying surfactant may disturb the good efficacy of the hydrophilic active agent, the latter possibly being less available to penetrate well into the skin.

The presence of a thickening polymer makes it possible to reduce or even dispense with the presence of emulsifying surfactants in the emulsions. But certain thickeners also present drawbacks. Hydroxyethyl cellulose forms a brittle and powdery film and in the presence of certain active agents such as salts and vitamins, the viscosity of the composition is greatly reduced. Xanthan or guar gums have a sticky feel or give a stringy appearance to the composition; the thickened composition takes a long time to penetrate during its application to the skin. Polyvinyl-pyrrolidone has a tendency to form a film that is too stiff and therefore is brittle. Thus, it is therefore difficult to find a good compromise between a good thickened texture of the composition and good application to keratin materials, especially to the skin, especially in order to allow good penetration of the composition and the active agents into the skin. In addition, it is desirable during spreading of the composition on the skin that the texture of the composition remains constant during the application without having a sudden change that generates a retardive sensation that is unpleasant for the user.

### SUMMARY OF THE INVENTION

One object of the present invention is therefore to provide a composition, of which the application to keratin materials, especially to the skin, is comfortable, gentle, without a retardive sensation during spreading, nor a sticky effect.

Another object of the invention is to provide a skincare composition enabling good penetration of the cosmetic or dermatological hydrophilic active agents into the skin and therefore having a good cosmetic efficacy (for example, a good anti-wrinkle efficacy).

The inventors have discovered that a composition meeting the above objectives, and providing further benefits, is obtained by formulating an oil-in-water dispersion in the presence of a mixture of particular thickeners.

### DETAILED DESCRIPTION OF THE PREFERRED EMBODIMENTS

In one preferred embodiment, the invention relates to a composition in the form of an oil-in-water dispersion comprising:
at least 70% by weight of water, relative to the total weight of the composition;
at least one glyceryl acrylate polymer;
at least one homopolymer of a monomer having a sulphonic group;
at least one volatile oil in an amount of less than or equal to 20% by weight, relative to the total weight of the composition; and
0 to 0.5% of at least one non-volatile oil.

All embodiments of the invention, including the one described above, is open to multiple components of the same general type—e.g., more than one glyceryl acrylate polymer. This is true for all embodiments of the invention, regardless whether the phrase “at least one” is used or the term, e.g., “a” is used in the description.

The compositions according to the invention, after application to the skin, have good cosmetic properties of softness and comfort. In addition, when the compositions contain a hydrophilic active agent, it contributes to good penetration of the active agent promoting a good cosmetic activity (for example, a good anti-wrinkle efficacy).

Another subject of the invention is a non-therapeutic keratin material care or makeup method that comprises the application of the composition defined previously onto keratin materials.

The composition according to the invention comprises at least one glyceryl acrylate polymer that is a hydrophilic thickener.

Preferably, the glyceryl acrylate polymer(s) is chosen from glyceryl acrylate/acrylic acid copolymers. Such copolymers are especially sold under the names LUBRAJEL® MS, LUBRAJEL® CG, LUBRAJEL® DV, LUBRAJEL® NP, LUBRAJEL® OII, LUBRAJEL® Oil BG, LUBRAJEL® PF, LUBRAJEL® TW, LUBRAJEL® WA by Guardian Laboratories. Preferably LUBRAJEL® MS is used.

The glyceryl acrylate polymer(s) may be present in the composition according to the invention in an amount ranging for example from 0.01% to 5% by weight, relative to the total weight of the composition, preferably ranging for example from 0.05% to 3% by weight, and more preferably ranging for example from 0.1 to 3% by weight, including 0.5, 1, 1.5, 2 and 2.5% by weight.

Advantageously, the composition according to the invention comprises a polymer having at least one monomer having a sulphonic group. The presence of this polymer enhances the cosmetic properties, in particular the absence of stringing, especially when the composition is taken up with the finger, absence of sticking, and softness, and also promotes good penetration of the composition when applied to the skin.

The polymers comprising at least one monomer having a sulphonic group, used in the composition of the invention, are preferably water-soluble or water-dispersible or swellable in water. The polymers used according to the invention are preferably homopolymers capable of being obtained from at least one ethylenically unsaturated monomer having a sulphonic group, which may be in free form or partially or completely neutralized form.

More preferably, the polymers according to the invention may be partially or completely neutralized by a mineral base (sodium hydroxide, potassium hydroxide, or ammonia) or an organic base such as mono-, di- or triethanolamine, an aminomethylpropanediol, N-methylglucamine, basic amino acids such as arginine and lysine, and mixtures of these compounds. They are preferably generally neutralized. The term “neutralized” is understood to mean in the present invention polymers that are completely or almost completely neutralized, that is to say at least 90% neutralized.

The polymers used in the composition of the invention generally preferably have a number-average molecular weight ranging for example from 1000 to 20 000 000 g/mol, preferably ranging for example from 20 000 to 5 000 000 and more preferably still from 100 000 to 1 500 000 g/mol.

These polymers according to the invention may be crosslinked or uncrosslinked.

The monomers having a sulphonic group for the polymer used in the composition of the invention are especially (i.e. preferably) chosen from vinylsulphonic acid, styrenesulphonic acid, (meth)acrylamido(C₁-C₂₂)alkyl-sulphonic acids, N—(C₁-C₂₂)-alkyl(meth)acrylamido (C₁-C₂₂)alkylsulphonic acids such as undecylacrylamidomethanesulphonic acid, and also partially or completely neutralized forms thereof and mixtures thereof.

According to one preferred embodiment of the invention, the monomers having a sulphonic group are chosen from (meth)acrylamido(C₁-C₂₂)alkylsulphonic acids such as for example acrylamidomethanesulphonic acid, acrylamido-ethanesulphonic acid, acrylamidopropanesulphonic acid, 2-acrylamido-2-methylpropanesulphonic acid, 2-meth-acrylamido-2-methylpropanesulphonic acid, 2-acrylamido-n-butanesulphonic acid, 2-acrylamido-2,4,4-trimethyl-pentanesulphonic acid, 2-methacrylamidododecylsulphonic acid, and 2-acrylamido-2,6-dimethyl-3-heptanesulphonic acid, and also partially or completely neutralized forms thereof and mixtures thereof.

More particularly, 2-acrylamido-2-methylpropane-sulphonic acid (AMPS) is used and also its partially or completely neutralized forms.

When the polymers are crosslinked, the crosslinking agents may be chosen from the polyolefinically unsaturated compounds, commonly used for crosslinking polymers obtained by radical polymerization.

As examples of crosslinking agents, mention may be made, without limitation, of: divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylene-bismethacrylamide, triallylamine, triallyl cyanurate, diallyl maleate, tetraallylethylenediamine, tetraallyloxyethane, trimethylolpropane diallyl ether, allyl(meth)acrylate, allyl ethers of alcohols from the sugar series, or other allyl or vinyl ethers of polyfunctional alcohols, and also allyl esters of phosphoric and/or vinylphosphonic acid derivatives, or mixtures of these compounds.

According to one preferred embodiment of the invention, the crosslinking agent is chosen from methylenebisacrylamide, allyl methacrylate or trimethylolpropane triacrylate (TMPTA). The degree of crosslinking generally ranges from 0.01 to 10 mol % and more particularly from 0.2 to 2 mol % relative to the polymer.

The homopolymer of monomers having a sulphonic group may be crosslinked with one or more crosslinking agents.

These homopolymers are generally crosslinked and neutralized, and they may be obtained according to the preparation process comprising the following steps:

(a) the monomer, such as 2-acrylamido-2-methylpropanesulphonic acid, is dispersed or dissolved in free form in a solution of tert-butanol or water and tert-butanol;

(b) the solution or dispersion of monomer obtained in (a) is neutralized by one or more mineral or organic bases, preferably ammonia NH₃, in an amount that makes it possible to obtain a degree of neutralization of the sulphonic acid functional groups of the polymer ranging for example from 90 to 100%;

(c) the crosslinking monomer or monomers are added to the solution or dispersion obtained in (b);

(d) a conventional radical polymerization is carried out in the presence of free-radical initiators at a temperature ranging for example from 10 to 150° C.; the polymer precipitating into the solution or dispersion based on tert-butanol.

Preferred AMPS homopolymers are in general characterized in that they comprise, randomly distributed:

a) from 90 to 99.9% by weight of units of general formula (II) below:

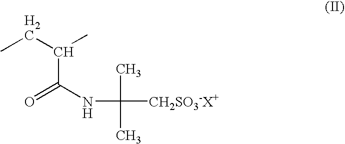

in which X⁺ denotes a proton, an alkali metal cation, an alkaline-earth metal cation or an ammonium ion, at most 10 mol % of X⁺ cations possibly being protons H⁺;

b) from 0.01 to 10% by weight of crosslinking units originating from at least one monomer having at least two olefinic double bonds; the weight proportions being defined relative to the total weight of the polymer.

The more particularly preferred homopolymers according to the invention comprise from 98 to 99.5% by weight of units of formula (II) and from 0.2 to 2% by weight of crosslinking units.

As polymers of this type, mention may especially be made of the crosslinked and neutralized homopolymer of 2-acrylamido-2-methylpropanesulphonic acid, sold by Clariant under the trade name Hostacerin AMPS (CTFA name: ammonium polyacryldimethyltauramide).

The homopolymer of a monomer having a sulphonic group may be present in the composition according to the invention in an amount of active material for example ranging for example from 0.01 to 10% by weight, preferably ranging for example from 0.1 to 5% by weight, more preferably ranging for example from 0.1 to 3% by weight, relative to the total weight of the composition.

Advantageously, the glyceryl acrylate polymer and the homopolymer of a monomer having a sulphonic group described previously may be present in the composition according to the invention in a glyceryl polyacrylate/homopolymer of a monomer having a sulphonic group weight ratio ranging for example from 1 to 4, preferably ranging for example from 1.5 to 3, more preferably ranging for example from 1.5 to 2.5 and more preferably still ranging for example from 1.8 to 2.3.

The composition according to the invention may comprise up to 20% by weight of volatile oil, relative to the total weight of the composition, especially in an amount ranging for example from 0.1% to 20% by weight, relative to the total weight of the composition, preferably ranging for example from 0.5% to 15% by weight, more preferably ranging for example from 1% to 10% by weight, and more preferably still ranging for example from 5% to 10% by weight. The volatile oil enables an easy and pleasant spreading of the composition on the skin.

The volatile oil may be chosen from silicone volatile oils or non-silicone volatile oils.

The term “volatile oil” is understood to mean any non-aqueous medium capable of evaporating on the skin or lips, in less than one hour, especially having a vapour pressure, at room temperature and atmospheric pressure, ranging for example from 1×10⁻³ to 300 mmHg (0.13 Pa to 40 000 Pa).

As the volatile oil that can be used in the invention, use can be made of the non-silicone volatile oils, especially C₈-C₁₆ isoparaffins such as isododecane, isodecane, isohexadecane and, for example, the oils sold under the commercial names ISOPAR, PERMETHYL and especially isododecane (PERMETHYL 99 A).

As the silicone volatile oil that can be used in the invention, mention may be made of the linear or cyclic silicones having from 2 to 7 silicon atoms, these silicones optionally comprising alkyl or alkoxy groups having from 1 to 10 carbon atoms. In particular, mention may be made of octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane, decamethyltetrasiloxane and mixtures thereof.

Advantageously, the composition according to the invention may comprise up to 0.5% (especially from 0.1 to 0.5%) by weight, relative to the total weight of the composition, of a surfactant having a HLB greater than or equal to 14, especially ranging for example from 14 to 18, and preferably ranging for example from 14 to 16. Such a surfactant does not help to emulsify the oily phase in the aqueous phase but makes it possible to dissolve one or more fragrances that may be present in the composition.

Advantageously, the composition according to the invention does not comprise an emulsifying surfactant having a HLB (hydrophilic/lipophilic balance) of less than 14, that is to say that such an emulsifier is present in an amount of less than 0.5% by weight, relative to the total weight of the composition, or even is absent (zero content) from the composition.

The HLB value according to Griffin is defined in J. Soc. Cosm. Chem., 1954 (volume 5), pages 249-256. Reference may be made to the document “Kirk-Othmer Encyclopedia of Chemical Technology”, volume 22, p. 333-432, 3^{rd} edition, 1979, Wiley, for the definition of the properties and (emulsifying) functions of surfactants, in particular p. 347-377 from this reference, for non-ionic surfactants.

As an example of a surfactant having a HLB greater than 14, mention may be made of fatty acid esters and polyethylene glycol esters such as ethoxylated hydrogenated castor oil, especially comprising 30 to 70 ethylene oxide units, such as those sold under the names CREMOPHOR® RH40 and CREMOPHOR® RH60 by BASF. Mention may also be made of ethoxylated nonylphenol, especially having 12 ethylene oxide units, such as that sold under the name SOLUBILISANT S 12 by Givaudan.

The composition according to the invention preferably contains less than 0.5% (or from 0 to 0.5% by weight) by weight of non-volatile oil, relative to the total weight of the composition, or is even free (zero content) of non-volatile oil.

The term “non-volatile oil” is understood to mean an oil capable of staying on the skin at room temperature (25° C.) and atmospheric pressure for at least one hour and especially having a non-zero vapour pressure at room temperature (25° C.) and atmospheric pressure that is less than 0.01 mmHg (1.33 Pa).

As non-volatile oils that can be used in the invention, mention may be made of:
non-silicone, especially hydrocarbon-based, non-volatile oils, such as liquid paraffin (or liquid petrolatum), squalane, hydrogenated polyisobutylene (Parleam oil), perhydrosqualene, mink, turtle or soybean oil, sweet almond, calophyllum, palm, grape seed, sesame, maize, arara, rapeseed, sunflower, cotton seed, apricot, castor, avocado, jojoba, olive or wheatgerm oil; esters of lanolic acid, oleic acid, lauric acid, or stearic acid; esters derived from long-chain (that is to say having 6 to 20 carbon atoms) acids or alcohols, especially esters of formula RCOOR′ in which R represents the residue of a higher fatty acid comprising 7 to 19 carbon atoms and R′ represents a hydrocarbon-based chain comprising 3 to 20 carbon atoms, in particular the C₁₂-C₃₆ esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or 2-octyldodecyl lactate, di(2-ethylhexyl)succinate, diisostearyl malate, glyceryl triisostearate or diglyceryl triisostearate; higher, especially C₁₄-C₂₂, fatty acids such as myristic acid, palmitic acid, stearic acid, behenic acid, oleic acid, linoleic acid, linolenic acid or isostearic acid; higher, especially C₁₆-C₂₂, fatty alcohols such as cetanol, oleyl alcohol, linoleyl alcohol or linolenyl alcohol, isostearyl alcohol or octyldodecanol; and mixtures thereof;
non-volatile silicone oils such as non-volatile polydimethylsiloxanes (PDMS); polydimethylsiloxanes comprising alkyl, alkoxy or phenyl groups, which are pendant or at the end of the silicone chain, the groups having from 2 to 24 carbon atoms; phenyl silicones such as phenyl trimethicones, phenyl dimethicones, phenyl trimethylsiloxydiphenylsiloxanes, diphenyl dimethicones, diphenyl methyldiphenyltrisiloxanes; polysiloxanes modified by fatty acids (especially C₈-C₂₀ fatty acids), by fatty alcohols (especially C₈-C₂₀ fatty alcohols) or by polyoxyalkylenes (especially polyoxyethylene and/or polyoxypropylene); amino silicones; silicones having hydroxyl groups; fluorosilicones comprising a fluoro group, which is pendant or at the end of the silicone chain, having from 1 to 12 carbon atoms, in which all or some of the hydrogens are substituted by fluorine atoms; and mixtures thereof.

The composition according to the invention comprises an aqueous phase.

The composition may comprise water preferably in an amount ranging for example from 60% to 95% by weight, relative to the total weight of the composition, more preferably ranging for example from 65% to 90% by weight, and even more preferably ranging for example from 70% to 80% by weight.

The water is not limited and may be a floral water such as cornflower water and/or a mineral water such as water from Vittel, water from Lucas or water from La Roche Posay and/or a thermal water.

The composition may, in addition, comprise at least one polyol that is miscible with water at room temperature (25° C.) especially chosen from polyols particularly having 2 to 20 carbon atoms, preferably having 2 to 10 carbon atoms and more preferably having 2 to 6 carbon atoms, such as glycerol, propylene glycol, butylene glycol, pentylene glycol, hexylene glycol, dipropylene glycol or diethylene glycol;

glycol ethers (especially having 3 to 16 carbon atoms) such as (C₁-C₄)alkyl ethers of mono-, di- or tripropylene glycol, and (C₁-C₄)alkyl ethers of mono-, di- or triethylene glycol; and mixtures thereof.

The composition according to the invention may comprise a polyol that is miscible with water at room temperature in an amount ranging for example from 1% to 20% by weight, relative to the total weight of the composition, and preferably ranging for example from 3% to 15% by weight.

Moreover, the composition according to the invention may comprise a monoalcohol having from 2 to 6 carbon atoms, such as ethanol or isopropanol, in an amount of less than or equal to 5% by weight, relative to the total weight of the composition, especially ranging for example from 0.01 to 5% by weight. According to one particular embodiment of the invention, the composition may be free of such a monoalcohol (zero content).

The composition may comprise fillers for example in an amount of less than or equal to 3% by weight, relative to the total weight of the composition, especially in an amount ranging for example from 0.1 to 3% by weight. According to one embodiment of the invention, the composition is free of fillers (zero content).

The term “filler” is understood to mean any colourless particle chosen from mineral or organic, lamellar, spherical or oblong fillers that are chemically inert in the composition.

Mention may be made of talc, mica, silica, kaolin, laponite, polyamide powders such as Nylon®, poly-p-alanine and polyethylene powders, powders of tetrafluoroethylene polymers (Teflon®), lauroyl lysine, starch, boron nitride, hollow polymer microspheres such as those made from polyvinylidene chloride/acrylonitrile such as Expancel® (Nobel Industrie), acrylic polymer particles, especially particles of acrylic acid copolymer such as Polytrap® (Dow Corning), polyurethane powders, silicone resin microbeads (Tospearls® from Toshiba, for example), precipitated calcium carbonate, dicalcium phosphate, magnesium carbonate and magnesium hydrogen carbonate, hydroxyapatite, hollow silica microspheres (Silica Beads® from Maprecos), glass or ceramic microcapsules, metallic soaps derived from organic carboxylic acids having from 8 to 22 carbon atoms, preferably from 12 to 18 carbon atoms, for example zinc, magnesium or lithium stearate, zinc laurate, magnesium myristate, and mixtures thereof.

The composition advantageously comprises a cosmetic or dermatological hydrophilic active agent. The hydrophilic active agent may be any water-soluble molecule having a cosmetic or dermatological activity and having a solubility in water of at least 0.25% by weight at room temperature (25° C.).

The hydrophilic active agents may especially be chosen from skin-relaxing agents, agents that stimulate the synthesis of fibres of the dermal or epidermal extracellular matrix or of fibres located at the dermal-epidermal junction and/or that prevent their degradation, anti-glycation agents, lipolytic active agents or active agents that inhibit lipogenesis, anti-irritants, and mixtures thereof. Such hydrophilic active agents are described, for example, in Application FR-A-2877221.

Mention may also be made, by way of example, of:
free-radical scavengers and/or detoxifying agents such as ascorbic acid and derivatives thereof such as magnesium ascorbyl phosphate, cysteine derivatives such as for example N-acetylcysteine, proteins and enzymes, for example superoxide dismutase (SOD), peroxydases such as lactoperoxydase and lactoferrin, catalase, proteases such as subtilisin and papain, lipases, uricase, peptides and derivatives thereof, ubiquinone and cytochrome C;
keratolytic agents such as a-hydroxy acids, β-hydroxy acids and α-keto acids, such as salicylic acid and derivatives thereof;
tanning accelerators such as tyrosine derivatives;
depigmenting active agents such as kojic acid, arbutin and derivatives thereof;
self-tanning active agents such as dihydroxyacetone and indoles;
lipid regulators such as caffeine and theophylline;
moisturizers such as sorbitol, xylitol, urea and plant DNA;
antidandruff agents such as piroctone olamine and pyridinethione derivatives;
anti-elastase agents, such as {2-[acetyl-(3-tri-fluoromethylphenyl)amino]-3-methylbutyrylamino}acetic acid (compound described in Application WO 01/94381);
skin-relaxing agents such as adenosine; and
mixtures thereof.

Preferentially, the hydrophilic active agent is chosen from {2-[acetyl-(3-trifluoromethylphenyl)amino]-3-methylbutyrylamino}acetic acid, adenosine, caffeine, and mixtures thereof.

The hydrophilic active agent or agents may be present in the composition according to the invention in an amount ranging for example from 0.001% to 10% by weight, preferably ranging for example from 0.01% to 5% by weight, and more preferably ranging for example from 0.05% to 1% by weight, relative to the total weight of the composition.

Advantageously, the composition according to the invention preferably contains less than 0.5% by weight of wax, relative to the total weight of the composition, or even is free of wax (zero content).

The term “wax” is understood to mean, in the sense of the present invention, a lipophilic compound that is solid at room temperature (25° C.), having a reversible solid/liquid phase change and having a melting point greater than or equal to 30° C. which may range up to 120° C.

The melting point of the wax may be measured using differential scanning calorimetry (DSC), for example the calorimeter sold under the name DSC 30 by Metler.

The composition according to the invention may also comprise the usual adjuvants in the cosmetics and dermatological fields, such as preservatives, antioxidants, fragrances, fillers, pigments, UV screening agents, odour absorbers and colorants. The amounts of these various adjuvants are those conventionally used in the fields in question, and for example are from 0.01 to 20% of the total weight of the composition.

Obviously, a person skilled in the art will be sure to choose the optional compound or compounds to add to the compositions according to the invention, and also their concentration, so that the advantageous properties intrinsically attached to the compositions according to the invention are not, or are not substantially, impaired by the envisaged addition.

The composition may be for example a care composition, especially a skincare product, such as a skincare base, a care cream (day cream, night cream, anti-wrinkle cream), a makeup base, a care composition for the lips (lip balm), a sunscreen or self-tanning composition.

The composition may also be a makeup composition, especially a skin makeup product, such as a foundation, face powder, eyeshadow, concealer product or a body makeup product.

The examples that follow illustrate the invention without limiting it.

### EXAMPLE 1

An anti-wrinkle face care composition was prepared containing the following ingredients:

| acetyl trifluoromethylphenyl valylglycine* | 0.08 | g |
|---|---|---|
| caffeine | 0.5 | g |
| mixture of Saccharomyces cerevisiae, mannitol, | 0.2 | g |
| cyclodextrin and disodium succinate (CYTOVITIN | | |
| LS 9388 from Laboratoires Serobiologiques) | | |
| adenosine | 0.04 | g |
| soya protein hydrolysate | 0.4 | g |
| (Phytokine ® from Coletica) | | |
| 5.5% extract of Lentinus Edodes in water | 0.4 | g |
| (Ferminskin ® from Silab) | | |
| polyacrylamidomethylpropanesulphonic acid | 1 | g |
| partially neutralized with ammonia and highly | | |
| crosslinked (Hostacerin AMPS from Clariant) | | |
| glyceryl acrylate/acrylic acid copolymer at 1% | 5 g i.e. 0.05 g AM | |
| in water/glycerol (LUBRAJEL ® MS from | | |
| Guardian Laboratories) | | |
| 20% fucose in water (Fucogel ® 1000 PP | 2 | g |
| from Solabia) | | |
| cyclopentasiloxane | 7 | g |
| mixture of cyclopentasiloxane and dimethiconol | 1 | g |
| (Dow Corning 1501 Fluid) | | |
| glycerol | 7 | g |
| propylene glycol | 2 | g |
| disodium salt of ethylenediaminetetraacetic | 0.15 | g |
| acid | | |
| triethanolamine | 0.05 | g |
| preservatives | qs | |
| water | qs for 100 | g |

*= {2-[acetyl-(3-trifluoromethylphenyl)amino]-3-methylbutyrylamino}acetic acid otherwise known as N-[N-acetyl, N′-(3-trifluoromethyl)phenylvalyl]glycine or N-acetyl-N-[3-(trifluoromethyl)phenyl]valylglycine or acetyl trifluoromethyl phenyl valylglycine, prepared as described in Patent EP 1 292 608 B1.

The composition applied to the face is comfortable, soft, and has a good anti-wrinkle efficacy.

The anti-wrinkle efficacy of this composition was measured on a panel of 37 women aged from 40 to 70 years old, having all skin types, and having wrinkles and fine lines in the crow's foot area.
Use of the product 2 times a day for 4 weeks.
Assessment of the number, the surface area, and the length of the wrinkles by impressions taken with silicone rubber sold under the name SILFLO® by Flexico Developments LTD, then image analysis on the principle of measuring the cast shadows created by illuminating the impression with a low-angled light.

Impressions taken of the crow's foot wrinkles after treating for 4 weeks and for 12 weeks, compared to T 0 have enabled the following parameters to be evaluated:

| | T 4 weeks/T 0 | T 12 weeks/T 0 |
|---|---|---|
| Total number of wrinkles | −9% | −9% |
| Total wrinkled surface area | −23% | −13% |
| Length of the total | −16% | −13% |
| wrinkled surface area | | |

The results obtained show that the composition makes it possible to reduce the number of wrinkles and the total wrinkled surface area, thus proving the anti-wrinkle efficacy of the composition.

The cosmetic quality of the composition was also assessed on a panel of 51 women aged from 40 to 70 years old, having all skin types, and having wrinkles and fine lines in the crow's foot area.
Use of the product 2 times a day for 4 weeks.
The women filled in a questionnaire regarding the subjective efficacy of the product and the responses obtained were the following:

| | T immediate | T 4 weeks |
|---|---|---|
| The skin appeared smoother | 92% | 98% |
| The skin appeared more comfortable | 92% | 98% |
| The skin appeared softer | 98% | 98% |

The results obtained show that the composition applied to the skin gives a smoother effect to the skin and also appreciable comfort and softness.

The above written description of the invention provides a manner and process of making and using it such that any person skilled in this art is enabled to make and use the same, this enablement being provided in particular for the subject matter of the appended claims, which make up a part of the original description and including a composition in the form of an oil-in-water dispersion comprising:
at least 60% by weight of water, relative to the total weight of the composition;
at least one glyceryl acrylate polymer;
at least one homopolymer of a monomer having a sulphonic group;
at least one volatile oil in an amount of less than or equal to 20% by weight, relative to the total weight of the composition; and
0 to 0.5% of at least one non-volatile oil.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition in the form of an oil-in-water dispersion comprising:
at least 60% by weight of water, relative to the total weight of the composition;
at least one glyceryl acrylate polymer;
at least one homopolymer of a monomer having a sulphonic group;
at least one volatile oil, in an amount of less than or equal to 20% by weight relative to the total weight of the composition; and
0 to 0.5% of at least one non-volatile oil.

2. A composition according to claim 1, comprising a glyceryl acrylate/acrylic acid copolymer.

3. A composition according to claim 1, wherein the at least one glyceryl acrylate polymer is present in an amount of 0.01% to 5% by weight, relative to the total weight of the composition.

4. A composition according to claim 1, comprising at least one homopolymer of a monomer having a sulphonic group chosen from vinylsulphonic acid, styrenesulphonic acid, (meth)acrylamido(C₁-C₂₂)alkylsulphonic acids, N-(C₁-C₂₂)alkyl(meth)acrylamido(C₁-C₂₂)alkylsulphonic acids, partially and completely neutralized forms thereof, and mixtures thereof.

5. A composition according to claim 1, comprising a homopolymer of 2-acrylamido-2-methylpropanesulphonic acid.

6. A composition according to claim 1, comprising a crosslinked and neutralized homopolymer of 2-acrylamido-2-methylpropanesulphonic acid.

7. A composition according to claim 1, wherein the at least one homopolymer of a monomer having a sulphonic group is present in an amount of 0.01 to 10% by weight, relative to the total weight of the composition.

8. A composition according to claim 1, wherein the at least one glyceryl acrylate polymer and the at least one homopolymer of a monomer having a sulphonic group are present in a glyceryl polyacrylate/homopolymer of a monomer having a sulphonic group weight ratio of 1 to 4.

9. A composition according to claim 1, wherein the at least one volatile oil is present in an amount of 0.1% to 20% by weight, relative to the total weight of the composition.

10. A composition according to claim 1, wherein said composition is free of non-volatile oil.

11. A composition according to claim 1, comprising water in an amount of 60% to 95% by weight, relative to the total weight of the composition.

12. A composition according to claim 1, further comprising at least one polyol that is miscible with water at room temperature.

13. A composition according to claim 12, wherein the at least one polyol is chosen from glycerol, propylene glycol, butylene glycol, pentylene glycol, hexylene glycol, dipropylene glycol, diethylene glycol, and mixtures thereof.

14. A composition according to claim 12, wherein the at least one polyol that is miscible with water at room temperature is present in an amount of 1% to 20% by weight, relative to the total weight of the composition.

15. A composition according to claim 1, further comprising at least one monoalcohol having from 2 to 6 carbon atoms, in an amount of less than or equal to 5% by weight relative to the total weight of the composition.

16. A composition according to claim 1, wherein said composition is free of a monoalcohol having from 2 to 6 carbon atoms.

17. A composition according to claim 1, further comprising one or more fillers, in an amount of less than or equal to 3% by weight relative to the total weight of the composition.

18. A composition according to claim 1, wherein said compositon is free of filler.

19. A composition according to claim 1, wherein said compositon comprises less than 0.5% by weight of wax, relative to the total weight of the composition.

20. A method comprising applying a composition according to claim 1 onto a keratin material.
